(19) United States
(12) Patent Application Publication
(10) Pub. No.: US 2021/0293346 A1
(43) Pub. Date: Sep. 23, 2021

(54) FLUID PATH OPENING/CLOSING DEVICE, FLOW RATE CONTROL DEVICE, AND BLOOD PRESSURE MONITOR

(71) Applicants: OMRON Corporation, Kyoto-shi, Kyoto (JP); OMRON HEALTHCARE Co., Ltd., Muko-shi, Kyoto (JP)

(72) Inventors: Toshifumi SUMINO, Kyoto-shi, Kyoto (JP); Yuki YAMAMOTO, Kyoto-shi, Kyoto (JP); Takehiro HAMAGUCHI, Muko-shi, Kyoto (JP); Yoshihiko SANO, Muko-shi, Kyoto (JP); Minoru TANIGUCHI, Muko-shi, Kyoto (JP); Chisato TAWARA, Muko-shi, Kyoto (JP)

(21) Appl. No.: 17/257,675

(22) PCT Filed: Feb. 6, 2019
(86) PCT No.: PCT/JP2019/004132

§ 371 Date: Jan. 4, 2021

(30) Foreign Application Priority Data
Jul. 3, 2018 (JP) 2018-126811

(51) Int. Cl. F16K 31/06; A61B 5/0235

(52) U.S. Cl. CPC F16K 31/0682; A61B 5/0235

(57) ABSTRACT

A fluid path opening/closing device includes a spool in a through-hole provided in an inside of the spool, a coil wound around the through-hole with respect to the spool, a movable iron piece accommodated in the through-hole in a state of being rotatable around a rotation axis, a yoke including a first member and a second member extending from one end portion in an extending direction of the first member toward the movable iron piece, the second member having an attraction portion that is capable of attracting and rotating the movable iron piece according to an energized state of the coil, and a fluid path unit including an opening portion provided to be closer to the attraction portion that to the rotation axis and provided near the attraction portion.

### TECHNICAL FIELD

The present disclosure relates to a fluid path opening/closing device, a flow control device including the fluid path opening/closing device, and a blood pressure monitor including the flow control device.

### BACKGROUND ART

Patent Literature 1 discloses a solenoid valve including a cylindrical fixed iron core, a solenoid coil arranged on the outer periphery of the fixed iron core, a nozzle portion including a discharge flow path, a valve body for closing the discharge flow path of the nozzle portion, and a rectangular plate-shaped movable member for movably supporting the valve body. In this solenoid valve, the valve body is provided at one end portion of the movable member in a longitudinal direction of the movable member, and a sucked portion to be sucked by the fixed iron core is provided at the other end portion of the movable member in the longitudinal direction. The movable member is configured to swing around a rotation axis arranged between the valve body and the sucked portion.

### CITATION LIST

### Patent Literature

PTL 1: JP 5364592 B2

### SUMMARY OF INVENTION

### Technical Problem

In the solenoid valve, since the sucked portion and the valve body are provided separately at both ends of the movable member in the longitudinal direction, the relative positional relationship between the sucked portion and the valve body tends to vary. Hence, it is difficult to close the discharge flow path with the valve body with higher accuracy in some cases. As a result, in a case where such a solenoid valve is used in a flow control device, the accuracy in the flow control of the fluid discharged from the discharge flow path may be reduced.

The present disclosure provides a fluid path opening/closing device capable of opening and closing an opening portion of a fluid path unit with high accuracy, a flow control device including the fluid path opening/closing device, and a blood pressure monitor including the flow control device.

### Solution to Problem

A fluid path opening/closing device as an example according to the present disclosure comprises:

a spool including a through-hole extending in a first direction, the through-hole being provided in an inside of the spool;

a coil wound around the through-hole with respect to the spool;

a movable iron piece extending in the first direction, the movable iron piece being accommodated in the through-hole in a state of being rotatable around a rotation axis;

a yoke including a first member extending in the first direction and a second member extending from one end portion in an extending direction of the first member toward the movable iron piece in a second direction intersecting the first direction, the first member being arranged outside the spool and being arranged to locate the coil between the movable iron piece and the first member in the second direction, the second member having an attraction portion that faces the movable iron piece in the second direction and that is capable of attracting and rotating the movable iron piece according to an energized state of the coil;

a fluid path unit including an opening portion provided to be closer to the attraction portion than to the rotation axis in the first direction and provided near the attraction portion, the opening portion being arranged to face the movable iron piece in the second direction, such that a fluid flows via the opening portion; and

a lid portion configured to be capable of opening and closing the opening portion through rotation of the movable iron piece. [0007]

A flow control device as an example according to the present disclosure includes:

the fluid path opening/closing device; and

a controller that control the energized state of the coil of the fluid path opening/closing device to open and close the opening portion and control a flow rate of the fluid flowing through the fluid path unit.

A blood pressure monitor as an example according to the present disclosure includes the flow control device.

### Advantageous Effects of Invention

The fluid path opening/closing device comprises the movable iron piece, the yoke, and the fluid path unit. The movable iron piece is accommodated in the through-hole of the spool in a state of being rotatable around the rotation axis. The yoke includes the attraction portion that is arranged to be capable of attracting and rotating the movable iron piece. The fluid path unit includes the opening portion that is arranged to be openable and closable through the rotation of the movable iron piece. The opening portion is provided closer to the attraction portion of the yoke than to the rotation axis of the movable iron piece and provided near the attraction portion. With such a configuration, since the opening portion of the fluid path unit is arranged to be closer to the attraction portion of the yoke than to the rotation axis of the movable iron piece, the relative positional relationship between the opening portion and the attraction portion is less likely to vary. Thus, the fluid path opening/closing device in which the opening portion of the fluid path unit can be opened and closed with high accuracy can be achieved.

According to the flow control device, the flow control device comprises the fluid path opening/closing device. Thus, the flow control device capable of controlling the flow rate of the fluid flowing through the fluid path unit via the opening portion with high accuracy can be achieved.

According to the blood pressure monitor, the blood pressure monitor comprises the flow control device. This eliminates the need for separately providing, for example, a mechanism for controlling the flow rate of the fluid flowing through the fluid path unit. Thus, the blood pressure monitor having a simple configuration can be achieved.

### BRIEF DESCRIPTION OF DRAWINGS

FIG. 1 is a block diagram of a blood pressure monitor comprising a fluid path opening/closing device according to an embodiment of the present disclosure.

FIG. 2 is a perspective view of the fluid path opening/closing device according to an embodiment of the present disclosure.

FIG. 3 is a cross-sectional view taken along line of FIG. 2.

FIG. 4 is a cross-sectional view taken along line IV-IV of FIG. 2.

FIG. 5 is a cross-sectional view showing a first modification of a fluid path opening/closing device of FIG. 2.

FIG. 6 is a cross-sectional view showing a second modification of the fluid path opening/closing device of FIG. 2.

FIG. 7 is a plan view of a fluid path opening/closing device of FIG. 6.

FIG. 8 is a cross-sectional view showing a third modification of the fluid path opening/closing device of FIG. 2.

FIG. 9 is a cross-sectional view showing a fourth modification of the fluid path opening/closing device of FIG. 2.

### DESCRIPTION OF EMBODIMENTS

Hereinafter, an example of the present disclosure will be described with reference to the accompanying drawings. It is to be noted that in the following description, terms indicating specific directions or positions (for example, terms including “upper”, “lower”, “right”, and “left”) are used as necessary, but use of those terms is for facilitating the understanding of the present disclosure with reference to the drawings, and the technical scope of the present disclosure is not limited by the meanings of the terms. In addition, the following description is merely an example and is not intended to limit the present disclosure, its application, or its use. Furthermore, the drawings are schematic, and the ratios of the respective dimensions and the like do not always match the actual ones.

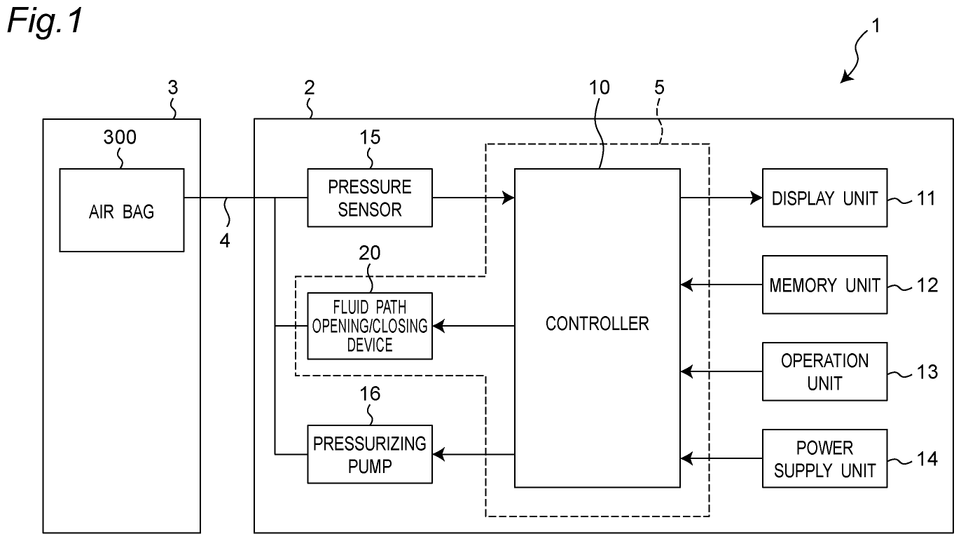

FIG. 1 shows a blood pressure monitor 1 including a fluid path opening/closing device 20 according to an embodiment of the present disclosure. The blood pressure monitor 1 includes a main body 2 and a cuff 3 including an air bag 300 in an inside thereof. The air bag 300 is connected with a pressurizing pump 16 of the main body 2, which will be described later, via an air pipe 4.

In addition to the fluid path opening/closing device 20, the main body 2 includes a controller 10 that controls the entire blood pressure monitor 1, a display unit 11 that displays a measured blood pressure, a memory unit 12 that stores the measured blood pressure, an operation unit 13 that turns on and off a power supply, a power supply unit 14 that supplies power to the controller 10, a pressure sensor 15 that measures a pressure in the air pipe 4, and the pressurizing pump 16 that supplies air to the air bag 300 via the air pipe 4. The pressure sensor 15 and the fluid path opening/closing device 20 are each connected with the air pipe 4 between the air bag 300 and the pressurizing pump 16. The main body 2 may include at least the fluid path opening/closing device 20 and the controller 10 that controls the fluid path opening/closing device 20, and other configurations may be optionally added or deleted.

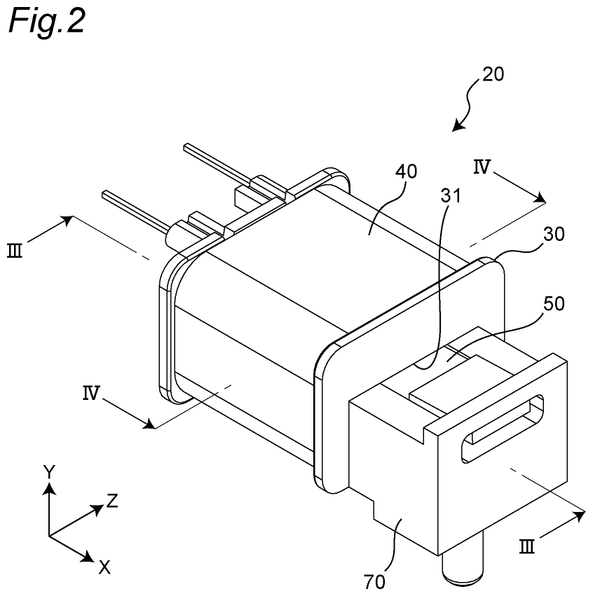

Next, the fluid path opening/closing device 20 will be described in more detail with reference to FIGS. 2 to 4.

As shown in FIGS. 2 to 4, the fluid path opening/closing device 20 includes a spool 30, a coil 40, a movable iron piece 50, a yoke 60, and a fluid path unit 70.

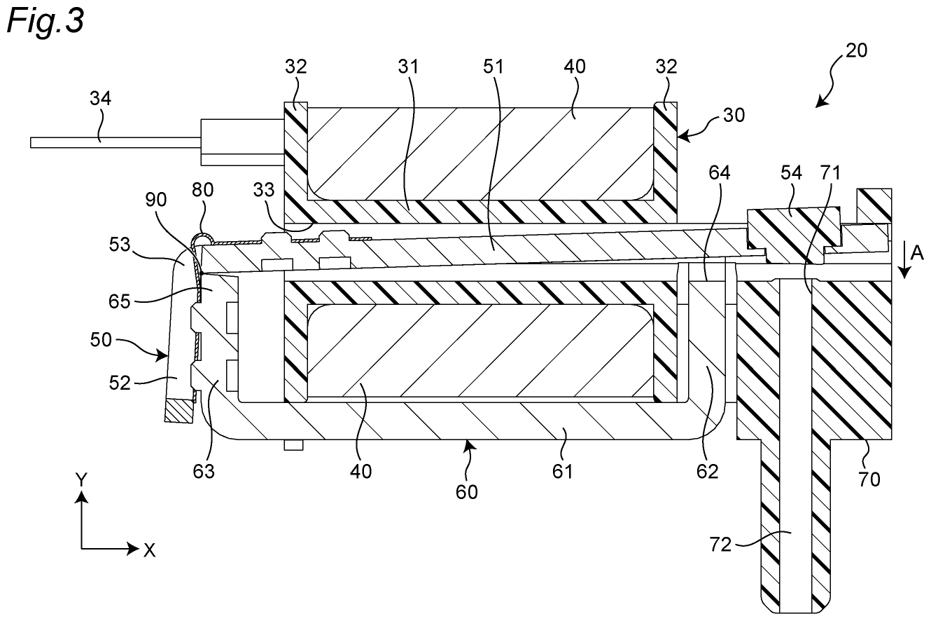

The spool 30, for example, is composed of an insulating resin. As shown in FIG. 3, the spool 30 includes a body portion 31 having a cylindrical shape and flange portions 32 provided at both ends in an extending direction of the body portion 31. The body portion 31 extends in a first direction X (that is, a left and right direction in FIG. 3). The flange portions 32 extend from the body portion 31 in a second direction Y intersecting (for example, orthogonal to) the first direction X. The body portion 31 has a through-hole 33 provided inside thereof. The through-hole 31 extends in the first direction X and penetrating through the body portion 31 in the first direction X. One of the flange portions 32 has a coil terminal 34 connected with the coil 40. Electric current of the coil 40 is supplied via the coil terminal 34.

The coil 40, for example, is composed of a copper magnet wire. The coil 40 is wound around the body portion 31 of the spool 30 as shown in FIG. 3. That is, the coil 40 is wound around the through-hole 33 with respect to the spool 30.

The movable iron piece 50, for example, is composed of a plate-shaped magnetic metal. As shown in FIG. 3, the movable iron piece 50 extends in the first direction X. The movable iron piece 50 is accommodated in the through-hole 33 of the spool 30 in a state of being rotatable around a rotation axis 90.

Specifically, the movable iron piece 50 has a substantially letter L shape when viewed from a third direction Z (that is, a paper penetration direction in FIG. 3) intersecting the first direction X and the second direction Y. The plate surface of the movable iron piece 50 is arranged to be orthogonal to the second direction Y. The movable iron piece 50 includes a first iron piece portion 51 and a second iron piece portion 52. The first iron piece portion 51 has a substantially rectangular plate shape and extends in the first direction X. The second iron piece portion 52 extends in the second direction Y from one end portion in an extending direction of the first iron piece portion 51 (that is, a left end part in FIG. 3). As an example, the first iron piece portion 51 and the second iron piece portion 52 are integrally provided.

The first iron piece portion 51 includes a rotation supported portion 53 provided at one end portion in the extending direction of the first iron piece portion 51. The rotation supported portion 53 constitutes the rotation axis 90 extending in the third direction Z. That is, the rotation supported portion 53 of the movable iron piece 50, which is arranged outside the spool 30 in the first direction X, is rotatably supported around the third direction Z by a rotation supporting portion 65 of the yoke 60. The first iron piece portion 51 includes a valve body 54 (an example of a lid portion) provided at the other end portion in the extending direction of the first iron piece 51 (a right end part in FIG. 3). The valve body 54 is configured to be capable of opening and closing an opening portion 71 of the fluid path unit 70, which will be described later, through the rotation of the movable iron piece 50. The valve body 54, for example, is composed of an elastic material such as rubber. The valve body 54 is arranged to face the opening portion 71 of the fluid path unit 70 in the second direction Y. That is, the valve body 54 is arranged to be capable of opening and closing the opening portion 71 of the fluid path unit 70 through the rotation of the movable iron piece 50.

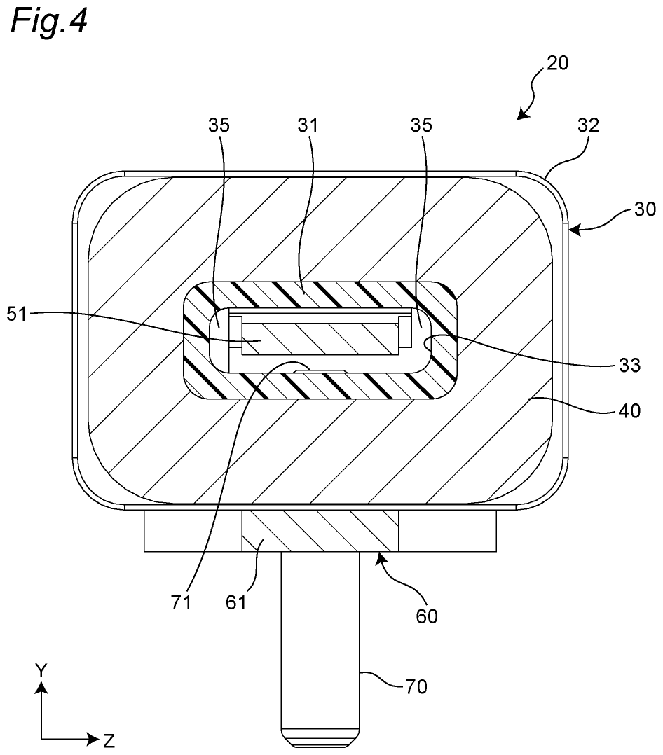

As shown in FIG. 4, a gap 35 is formed between the movable iron piece 50 and the through-hole 33 of the spool 30 in the third direction Z. With such a configuration, the movable iron piece 50 is capable of rotating more reliably around the rotation axis 90, while ensuring the allowable range of a processing error of the movable iron piece 50.

The yoke 60, for example, is composed of a plate-shaped magnetic metal. As shown in FIG. 3, the yoke 60 has a substantially letter U shape when viewed from the third direction Z and surrounds a part of the coil 40 together with the movable iron piece 50 around the third direction Z. The yoke 60 extends in the first direction X, and includes a first member 61, a second member 62, and a third member 63. The first member 61 is arranged outside the spool 30 in the second direction Y, and is arranged to locate the coil 40 between the movable iron piece 50 and the first member 61. The second member 62 extends from one end portion in an extending direction of the first member 61 (that is, the right end part in FIG. 3) toward the movable iron piece 50 in the second direction Y. The third member 63 extends from the other end portion in the extending direction of the first member 61 (that is, the left end part in FIG. 3) toward the movable iron piece 50 in the second direction Y. The second member 62 and the third member 63 are arranged outside the spool 30 and arranged near the flange portions 32 of the spool 30, respectively. As an example, the first member 61, the second member 62, and the third member 63 are integrally provided.

The second member 62 includes an attraction portion 64 provided at an end far from the first member 61 in the second direction Y of the second member 62. The attraction portion 64 is arranged to face the movable iron piece 50 in the second direction Y. The attraction portion 64 has a substantially planar shape including the first direction X and the third direction Y. The attraction portion 64 is arranged to be capable of attracting and rotating the movable iron piece 50 according to an energized state of the coil 40.

The third member 63 includes the rotation supporting portion 65 provided at an end far from the first member 61 in the second direction Y of the third member 63. The rotation-supporting portion 65 rotatably supports the movable iron piece 50 at the rotation-supported portion 53 of the movable iron piece 50. The rotation-supporting portion 65 has a substantially planar shape including the first direction X and the third direction Y. The rotation-supporting portion 65 is tilted to be closer to the first member 61 as approaching the flange portions 32 of the spool 30 in the first direction X. The rotation-supporting portion 65 is in contact with the rotation-supported portion 53 of the movable iron piece 50 at an end far from the flange portion 32 in the first direction X of the spool 30. That is, the movable iron piece 50 rotates around a part where the rotation-supported portion 53 is in contact with the rotation-supporting portion 65 of the yoke 60, as the rotation axis 90.

The movable iron piece 50 and the yoke 60 are connected with each other via a hinge spring 80. The hinge spring 80 is connected with one end portion in the extending direction of the first iron piece portion 51 of the movable iron piece 50 and the third member 63 of the yoke 60. In the second direction Y, the valve body 54 of the movable iron piece 50 urges the movable iron piece 50 in a direction separated from the opening portion 71 of the fluid path 70 (that is, upward in FIG. 3).

The fluid path unit 70, for example, is composed of an insulating resin. As shown in FIG. 3, the fluid path unit 70 includes the opening portion 71 provided to be closer to the attraction portion 64 of the yoke 60 than to the rotation axis 90 of the movable iron piece 50 in the first direction X and provided near the attraction portion 64. Specifically, the fluid path unit 70 is provided separately from the spool 30, and is arranged outside the spool 30 such that the second member 62 of the yoke 60 is located between the fluid path unit 70 and the flange portion 32 of the spool 30, in the first direction X.

The opening portion 71 is arranged to face the movable iron piece 50 in the second direction Y, and can be opened and closed by the valve body 54 through the rotation of the movable iron piece 50. As an example, the opening portion 71 is connected with a fluid path 72 through which a fluid flows, the fluid path 72 extending in the second direction Y. That is, the fluid path unit 70 is configured so that the fluid flows via the opening portion 71. An annular protrusion slightly protruding toward the movable iron piece 50 is formed on a peripheral edge of the opening portion 71. This protrusion allows the valve body 54 to close the opening portion 71 easily.

In the fluid path opening/closing device 20 in a non-energized state where no electric current is supplied to the coil 40, as shown in FIG. 3, the valve body 54 of the movable iron piece 50 is urged by the hinge spring 80 in a direction separated from the opening portion 71 of the fluid path 70 in the second direction Y, and the opening portion 71 is opened.

When electric current is supplied to the coil 40 via the coil terminal 34, a magnetic flux flows through the movable iron piece 50 and the yoke 60, and a magnetic circuit is formed. Accordingly, the movable iron piece 50 is attracted by the attraction portion 64 of the yoke 60 and rotates in the second direction Y and in a direction approaching the opening portion 71 of the fluid path unit 70 against the urging force of the hinge spring 80 (that is, a direction of an arrow A in FIG. 3), and the opening portion 71 is closed by the valve body 54.

In the present embodiment, the controller 10 and the fluid path opening/closing device 20 constitute a flow control device 5. The controller 10 includes a CPU that performs calculations, and a ROM and a RAM that store programs, data, and the like necessary for controlling the blood pressure monitor 1. The controller 10 controls the energized state of the coil 40 of the fluid path opening/closing device 20, so that the valve body 54 of the movable iron piece 50 in the second direction Y is brought into contact with or is separated from the opening portion 71 of the fluid path unit 70 to open or close the opening portion 71. As a result, the flow rate of the fluid flowing through the fluid path unit 70 via the opening portion 71 is controlled, to adjust the amount of air in the air bag 300.

The fluid path opening/closing device 20 includes the movable iron piece 50, the yoke 60, and the fluid path unit 70. The movable iron piece 50 is accommodated in the through-hole 33 of the spool 30 in a state of being rotatable around the rotation axis 90. The yoke 60 includes the attraction portion 64 that is arranged to be capable of attracting and rotating the movable iron piece 50. The fluid path unit 70 includes the opening portion 71 that is arranged to be openable and closable through the rotation of the movable iron piece 50. The opening portion 71 is provided closer to the attraction portion 64 of the yoke 60 than to the rotation axis 90 of the movable iron piece 50 and provided near the attraction portion 64. With such a configuration, since the opening portion 71 of the fluid path unit 70 is arranged to be closer to the attraction portion 64 of the yoke 60 than to the rotation axis 90 of the movable iron piece 50, the relative positional relationship between the opening portion 71 and the attraction portion 64 is less likely to vary. Accordingly, the fluid path opening/closing device 20 capable of opening and closing the opening portion 71 of the fluid path unit 70 with high accuracy can be achieved.

The movable iron piece 50 includes the rotation supported portion 53 provided at one end portion in the extending direction of the movable iron piece 50, the rotation supported portion 53 constituting the rotation axis 90. The rotation-supporting portion 65 that rotatably supports the movable iron piece 50 with the rotation-supported portion 53 is provided in the third member 63 of the yoke 60. Since the rotation-supporting portion 65 of the yoke 60 rotatably supports the movable iron piece 50, the opening portion 71 of the fluid path unit 70 can be opened and closed with higher accuracy.

The movable iron piece 50 includes the valve body 54 that faces the opening portion 71 of the fluid path unit 70 in the second direction Y. The valve body 54 is arranged to be capable of opening and closing the opening portion 71 through the rotation of the movable iron piece 50. With the valve body 54, the opening portion 71 of the fluid path unit 70 can be closed more reliably.

The opening portion 71 of the fluid path unit 70 is arranged to be adjacent to the attraction portion 64 in the first direction X at the outside of the spool 30. With such a configuration, the opening portion 71 of the fluid path unit 70 can be opened and closed with higher accuracy.

The flow control device 5 includes the fluid path opening/closing device 20, and the controller 10. The controller 10 controls the energized state of the coil 40 of the fluid path opening/closing device 20 to open and close the opening portion 71 and controls the flow rate of the fluid flowing through the fluid path unit 70. With such a configuration, the flow control device 5 capable of controlling the flow rate of the fluid flowing through the fluid path unit 70 via the opening portion 71 with high accuracy can be achieved.

The blood pressure monitor 1 includes the flow control device 5. This eliminates the need for separately providing, for example, a mechanism for controlling the flow rate of the fluid flowing through the fluid path unit 70. Therefore, a blood pressure monitor having a simple configuration can be achieved.

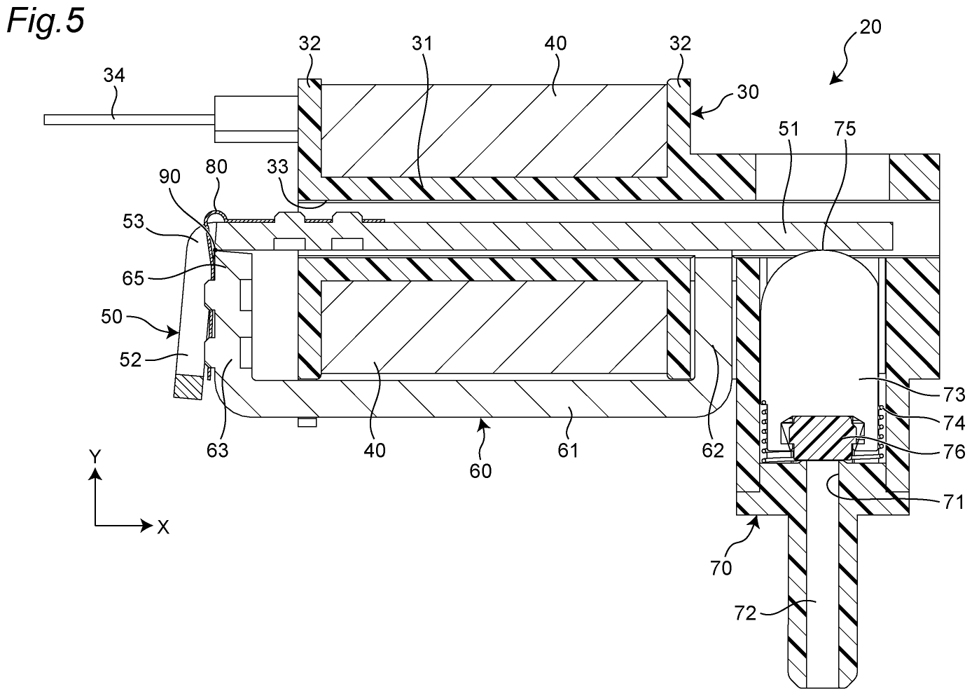

In the fluid path opening/closing device 20, the valve body 54 is provided in the movable iron piece 50, however, the present disclosure is not limited to this. For example, as shown in FIG. 5, the fluid path unit 70 may include a plunger 73 having a valve portion 76 and an urging portion 74. The plunger 73 is arranged in a moving direction between the movable iron piece 50 and the opening portion 71 in the second direction Y. The urging portion 74 (for example, a coil spring) urges the plunger 73 in the second direction Y toward the movable iron piece 50.

In the fluid path opening/closing device 20 shown in FIG. 5, the plunger 73 has a substantially cylindrical shape extending in the second direction Y, and includes a contact portion 75 provided to be contactable with the other end portion in the extending direction of the movable iron piece 50. A convex spherical surface protruding toward the movable iron piece 50 in the second direction Y is formed at an end of the plunger 73 on the contact portion 75 side, and the contact portion 75 is arranged at the apex of the convex spherical surface in the second direction Y. The valve portion 76 is arranged to face the opening portion 71 of the fluid path unit 70 and to be capable of opening and closing the opening portion 71 through a movement of the plunger 73 in the second direction Y. FIG. 5 shows the fluid path opening/closing device 20 in the energized state.

In this manner, the fluid path opening/closing device 20 may also be configured such that the opening portion 71 of the fluid path unit 70 is opened and closed via the plunger 73, which is an example of the lid portion, arranged in the moving direction between the movable iron piece 50 and the opening portion 71 in the second direction Y. Since the fluid path opening/closing device 20 includes the urging portion 74, the hinge spring 80 may be omitted. That is, the fluid path opening/closing device 20 having a high degree of freedom in design can be achieved.

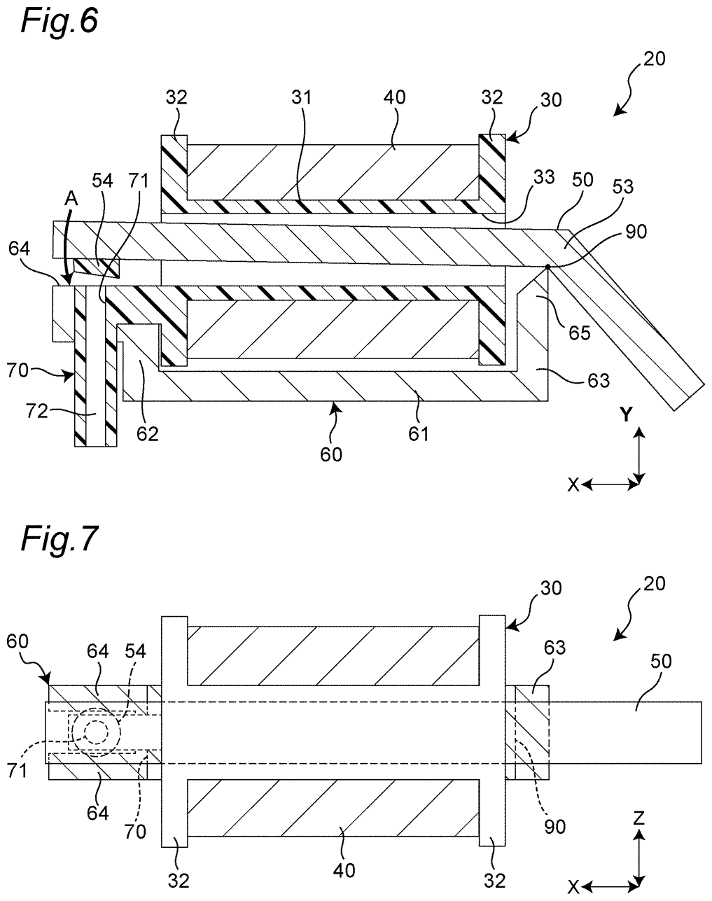

In the fluid path opening/closing device 20, the spool 30 and the fluid path unit 70 are provided separately, however, the present disclosure is not limited to this. For example, as shown in FIGS. 6 and 7, the spool 30 and the fluid path unit 70 may be provided integrally. In the fluid path opening/closing device 20 of FIGS. 6 and 7, the attraction portions 64 are provided in the second member 62 of the yoke 60. Each of the attraction portions 64 extend in the first direction X and a direction separated from the second member 62 from both end portions in the third direction Z, the attraction portions 64 being arranged to sandwich the opening portion 71 in the second direction Y.

By integrally providing the spool 30 and the fluid path unit 70 in this manner, the fluid path unit 70 can be easily positioned with respect to the spool 30. Therefore, the opening portion 71 of the fluid path unit 70 can be opened and closed with higher accuracy. Further, since the opening portion 71 of the fluid path unit 70 can be provided to be closer to the attraction portion 64, the fluid path opening/closing device 20 can be downsized.

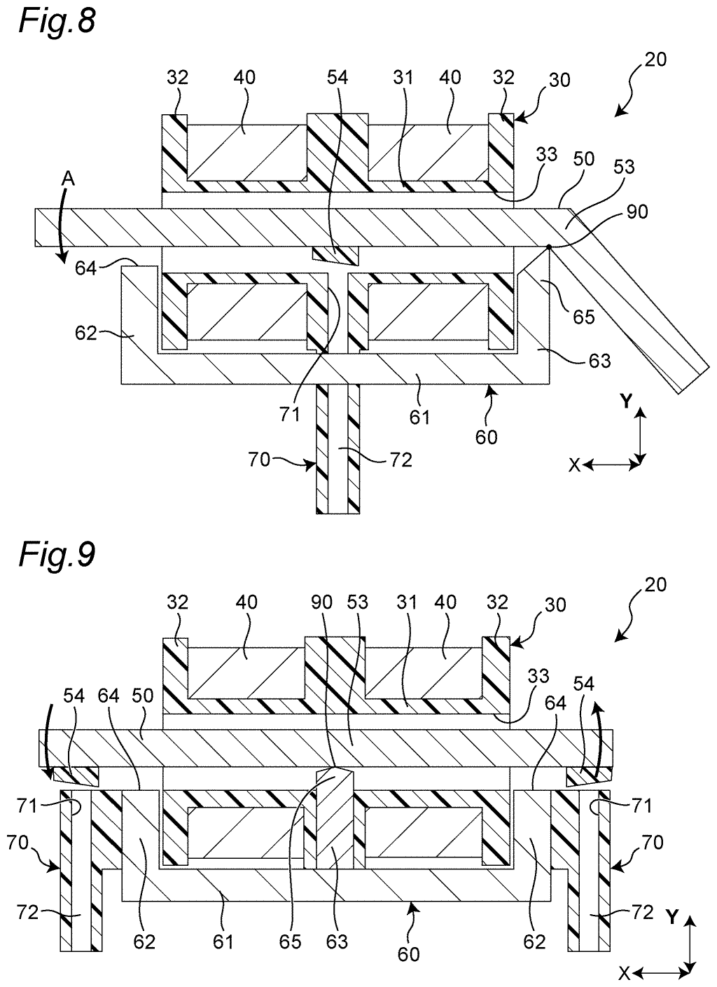

In a case where the spool 30 and the fluid path unit 70 are integrally provided, for example, as shown in FIG. 8, the opening portion 71 of the fluid path unit 70 may be provided inside the spool 30 in the first direction X. In the fluid path opening/closing device 20 of FIG. 8, two coils 40 are arranged side by side in the first direction X, and the opening portion 71 is arranged between these two coils 40. In this manner, the position of the opening portion 71 of the fluid path unit 70 may be appropriately changed according to the design of the device (for example, the blood pressure monitor 1) in which the fluid path opening/closing device 20 is provided. That is, the fluid path opening/closing device 20 having a high degree of freedom in design can be achieved.

In the fluid path opening/closing device 20, the rotation-supported portion 53 constituting the rotation axis 90 of the movable iron piece 50 is arranged outside the spool 30, however, the present disclosure is not limited to this. For example, as shown in FIG. 9, the rotation supported portion 53 may be provided inside the spool 30, that is, inside the through-hole 33. In the fluid path opening/closing device 20 of FIG. 9, the two coils 40 are arranged side-by-side in the first direction X, and the third member 63 composed of a permanent magnet is provided between these two coils 40 in the first direction X. In the fluid path opening/closing device 20 of FIG. 9, the fluid path units 70 are provided at both end portions of the fluid path opening/closing device 20 in the first direction X, and the valve bodies 54 are provided at both end portions of the movable iron piece 50 in the first direction X. The second members 62 are provided at both end portions of the yoke 60 in the first direction X.

In this manner, the position of the rotation axis 90 of the movable iron piece 50 may be appropriately changed according to the design of the device (for example, the blood pressure monitor 1) in which the fluid path opening/closing device 20 is provided. That is, the fluid path opening/closing device 20 having a high degree of freedom in design can be achieved.

In the fluid path opening/closing device 20 of FIG. 9, in both the energized state and the non-energized state, one opening portion 71 of the two fluid path units 70 is closed and the other opening portion 71 is opened. For example, by reversing a direction of the electric current flowing through the coil 40 and reversing the magnetizing magnetism of the movable iron piece 50, the state of one of the opening portions 71 can be changed from the normally open state to the normally closed state.

The movable iron piece 50 is not limited to the case of being rotatably supported by the third member 63 of the yoke 60. For example, a rotation-supporting portion that rotatably supports the rotation-supported portion 53 of the movable iron piece 50 may be provided in the spool 30. When the rotation-supporting portion is provided in the spool 30, the third member 63 of the yoke 60 may be omitted, if possible.

The valve body 54 may be omitted. In this case, the other end portion in the extending direction of the first iron piece portion 51 on the plate surface of the movable iron piece 50 constitutes the lid portion configured to be capable of opening and closing the opening portion 71 through the rotation of the movable iron piece 50.

In the fluid path opening/closing device 20, the movable iron piece 50 and the yoke 60 are formed in a substantially rectangular plate shape to increase the magnetic efficiency of the magnetic circuit formed in the movable iron piece 50 and the yoke 60, however, the present disclosure is not limited to this. Any shape can be adopted for the movable iron piece 50 and the yoke 60, as long as the opening portion 71 of the fluid path unit 70 can be opened and closed by the lid portion by controlling the energized state of the coil 40.

The fluid path opening/closing device 20 may be used as an electromagnetic relay by providing, for example, a conductive movable contact portion in place of the valve body 54 and a conductive fixed contact portion in place of the opening portion 71 of the fluid path unit 70.

Although various embodiments of the present disclosure have been described above in detail with reference to the drawings, finally, various aspects of the present disclosure will be described. In the following description, reference numerals are also attached as examples.

A fluid path opening/closing device 20 according to a first aspect of the present disclosure comprises:

a spool 30 including a through-hole 33 extending in a first direction X, the through-hole 33 being provided in an inside of the spool 30;

a coil 40 wound around the through-hole 33 with respect to the spool 30;

a movable iron piece 50 extending in the first direction X, the movable iron piece 50 being accommodated in the through-hole 33 in a state of being rotatable around a rotation axis 90;

a yoke 60 including a first member 61 extending in the first direction X and a second member 62 extending from one end portion in an extending direction of the first member 61 toward the movable iron piece 50 in a second direction Y intersecting the first direction X, the first member 61 being arranged outside the spool 30 and being arranged to locate the coil 40 between the movable iron piece 50 and the first member 61 in the second direction Y, the second member 62 having an attraction portion 64 that faces the movable iron piece 50 in the second direction Y and that is capable of attracting and rotating the movable iron piece 50 according to an energized state of the coil 40;

a fluid path unit 70 including an opening portion 71 provided to be closer to the attraction portion 64 than to the rotation axis 90 in the first direction X and provided near the attraction portion 64, the opening portion 71 being arranged to face the movable iron piece 50 in the second direction Y and being capable of opening and closing through rotation of the movable iron piece 50, such that a fluid flows via the opening portion 71.

The fluid path opening/closing device 20 according to the first aspect includes the movable iron piece 50, the yoke 60, and the fluid path unit 70. The movable iron piece 50 is accommodated in the through-hole 33 of the spool 30 in a state of being rotatable around the rotation axis 90. The yoke 60 includes the attraction portion 64 that is arranged to be capable of attracting and rotating the movable iron piece 50. The fluid path unit 70 includes the opening portion 71 that is arranged to be openable and closable through the rotation of the movable iron piece 50. The opening portion 71 is provided closer to the attraction portion 64 of the yoke 60 than to the rotation axis 90 of the movable iron piece 50 and provided near the attraction portion 64. With such a configuration, since the opening portion 71 of the fluid path unit 70 is arranged to be closer to the attraction portion 64 of the yoke 60 than to the rotation axis 90 of the movable iron piece 50, the fluid path opening/closing device 20 in which the opening portion 71 of the fluid path unit 70 can be opened and closed with high accuracy can be achieved.

In the fluid path opening/closing device 20 according to a second aspect of the present disclosure,

the movable iron piece 50 includes a rotation supported portion 53 which constitutes the rotation axis 90, the rotation supported portion 53 being provided at one end portion in an extending direction of the moveable iron piece 50,

the yoke 60 includes a third member 63 extending from another end portion in the extending direction of the first member 61 toward the movable iron piece 50 in the second direction Y, the third member 63 having a rotation supporting portion 65 that rotatably supports the movable iron piece 50 with the rotation supported portion 53.

According to the fluid path opening/closing device 20 according to the second aspect, since the rotation-supporting portion 65 of the yoke 60 rotatably supports the movable iron piece 50, the opening portion 71 of the fluid path unit 70 can be opened and closed with higher accuracy.

In the fluid path opening/closing device 20 according to a third aspect of the present disclosure,

the movable iron piece 50 includes a valve body 54 provided at another end portion in the extending direction of the moveable iron piece 50, the valve body 54 being arranged to face the opening portion 71 of the fluid path unit 70 in the second direction Y and to be capable of opening and closing the opening portion 71 through the rotation of the movable iron piece 50.

According to the fluid path opening/closing device 20 according to the third aspect, the valve body 54 can close the opening portion 71 of the fluid path unit 70 more reliably.

In the fluid path opening/closing device 20 according to a fourth aspect of the present disclosure,

the fluid path unit 70 includes

a plunger 73 arranged in a moving direction between the movable iron piece 50 and the opening portion 71 in the second direction Y, and

an urging portion 74 that urges the plunger 73 toward the movable iron piece 50 in the second direction Y,

the plunger 73 includes

a contact portion 75 arranged to be contactable with another end portion in the extending direction of the movable iron piece 50; and

a valve portion 76 that faces the opening portion 71 and that is arranged to be capable of opening and closing the opening portion 71 through a movement of the plunger 73 in the second direction Y.

According to the fluid path opening/closing device 20 according to the fourth aspect, the fluid path opening/closing device 20 having a high degree of freedom in design can be achieved.

In the fluid path opening/closing device 20 according to a fifth aspect of the present disclosure,

the opening portion 71 of the fluid path unit 70 is arranged to be adjacent to the attraction portion 64 in the first direction X at the outside of the spool 30.

According to the fluid path opening/closing device 20 according to the fifth aspect, the opening portion 71 of the fluid path unit 70 can be opened and closed with higher accuracy.

In the fluid path opening/closing device 20 according to a sixth aspect of the present disclosure,

the fluid path unit 70 is integrally provided with the spool 30.

According to the fluid path opening/closing device 20 according to the sixth aspect, since the fluid path unit 70 can be easily positioned with respect to the spool 30, the opening portion 71 of the fluid path unit 70 can be opened and closed with higher accuracy.

A flow control device 5 according to a seventh aspect of the present disclosure includes

the fluid path opening/closing device 20 of the above aspect, and

a controller 10 that controls the energized state of the coil 40 of the fluid path opening/closing device 20 and control a flow rate of the fluid flowing through the fluid path unit 70 via the opening portion 71.

According to the flow control device 5 according to the seventh aspect, since the fluid path opening/closing device 20 is provided, the flow control device 5 capable of controlling the flow rate of the fluid flowing through the fluid path unit 70 via the opening portion 71 with high accuracy can be achieved.

The blood pressure monitor 1 according to an eighth aspect of the present disclosure includes the flow control device 5 of the above aspect.

According to the blood pressure monitor 1 according to the eighth aspect, the flow control device 5 is provided. This eliminates the need for separately providing, for example, a mechanism for controlling the flow rate of the fluid flowing through the fluid path unit 70. Therefore, the blood pressure monitor having a simple configuration can be achieved.

It is to be noted that by appropriately combining any embodiment or modification from among the above described various embodiments or modifications, their effects can be brought out. In addition, any combination of embodiments, any combination of examples, and any combination of embodiments and examples are available, and features in different embodiments or examples are also available.

While the present disclosure has been fully described in connection with the preferred embodiments with reference to the accompanying drawings, various variations and modifications will be apparent to those skilled in the art. It is to be understood that such variations and modifications are intended to be included without departing from the scope of the present disclosure by the appended claims.

### INDUSTRIAL APPLICABILITY

The fluid path opening/closing device in the present disclosure can be used, for example, in a flow control device.

The flow control device in the present disclosure can be used, for example, in a blood pressure monitor.

The blood pressure monitor in the present disclosure can be used, for example, for an upper arm blood pressure monitor, a wrist blood pressure monitor, or a foot blood pressure monitor.

### REFERENCE SIGNS LIST

1. blood pressure monitor
2. main body
3. cuff
300. air bag
4. air pipe
5. flow control device
10. controller
11. display unit
12. memory unit
13. operation unit
14. power supply unit
15. pressure sensor
16. pressurizing pump
20. fluid path opening/closing device
30. spool
31. body portion
32. flange portion
33. through-hole
34. coil terminal
35. gap
40. coil
50. movable iron piece
51. first iron piece portion
52. second iron piece portion
53. rotation-supported portion
54. valve body
60. yoke
61. first member
62. second member
63. third member
64. attraction portion
65. rotation-supporting portion
70. fluid path
71. opening portion
72. fluid path
73. plunger
74. urging portion
75. contact portion
76. valve portion
80. hinge spring
90. rotation axis
A. arrow
X. first direction
Y. second direction
Z. third direction

1. A fluid path opening/closing device comprising:
a spool including a through-hole extending in a first direction, the through-hole being provided in an inside of the spool;
a coil wound around the through-hole with respect to the spool;
a movable iron piece extending in the first direction, the movable iron piece being accommodated in the through-hole in a state of being rotatable around a rotation axis;
a yoke including a first member extending in the first direction and a second member extending from one end portion in an extending direction of the first member toward the movable iron piece in a second direction intersecting the first direction, the first member being arranged outside the spool and being arranged to locate the coil between the movable iron piece and the first member in the second direction, the second member having an attraction portion that faces the movable iron piece in the second direction and that is capable of attracting and rotating the movable iron piece according to an energized state of the coil;
a fluid path unit including an opening portion provided to be closer to the attraction portion than to the rotation axis in the first direction and provided near the attraction portion, the opening portion being arranged to face the movable iron piece in the second direction, such that a fluid flows via the opening portion; and
a lid portion configured to be capable of opening and closing the opening portion through rotation of the movable iron piece.

2. The fluid path opening/closing device according to claim 1, wherein
the movable iron piece includes a rotation supported portion which constitutes the rotation axis, the rotation supported portion being provided at one end portion in an extending direction of the moveable iron piece, and
the yoke includes a third member extending from another end portion in the extending direction of the first member toward the movable iron piece in the second direction, the third member having a rotation supporting portion that rotatably supports the movable iron piece with the rotation supported portion.

3. The fluid path opening/closing device according to claim 2, wherein
the movable iron piece includes a valve body provided at another end portion in the extending direction of the moveable iron piece, the valve body being arranged to face the opening portion of the fluid path unit in the second direction and to be capable of opening and closing the opening portion through the rotation of the movable iron piece, and
the valve body constitutes the lid portion.

4. The fluid path opening/closing device according to claim 2, wherein
the fluid path unit includes:
a plunger arranged between the movable iron piece and the opening portion in the second direction; and
an urging portion that urges the plunger toward the movable iron piece in the second direction,
the plunger includes:
a contact portion arranged to be contactable with another end portion in the extending direction of the movable iron piece; and
a valve portion that faces the opening portion and that is arranged to be capable of opening and closing the opening portion through a movement of the plunger in the second direction, and
the plunger constitutes the lid portion.

5. The fluid path opening/closing device according to claim 3, wherein
the opening portion of the fluid path unit is arranged to be adjacent to the attraction portion in the first direction at an outside of the spool.

6. The fluid path opening/closing device according to claim 1, wherein
the fluid path unit is integrally provided with the spool.

7. A flow control device comprising:
the fluid path opening/closing device according to claim 1; and
a controller that controls the energized state of the coil of the fluid path opening/closing device to open and close the opening portion and control a flow rate of the fluid flowing through the fluid path unit.

8. A blood pressure monitor comprising the flow control device according to claim 7.

9. The fluid path opening/closing device according to claim 4, wherein the opening portion of the fluid path unit is arranged to be adjacent to the attraction portion in the first direction at an outside of the spool.

10. The fluid path opening/closing device according to claim 2, wherein the fluid path unit is integrally provided with the spool.

11. The fluid path opening/closing device according to claim 3, wherein the fluid path unit is integrally provided with the spool.

12. The fluid path opening/closing device according to claim 4, wherein the fluid path unit is integrally provided with the spool.
